(19) United States
(12) Patent Application Publication
(10) Pub. No.: US 2004/0138693 A1
(43) Pub. Date: Jul. 15, 2004

(54) Snare retrievable embolic protection filter with guidewire stopper

(75) Inventors: Alan D. Eskuri, Hanover, MN (US); Anthony C. Vrba, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Scimed Life Systems, Inc.

(21) Appl. No.: 10/341,667

(22) Filed: Jan. 14, 2003

(51) Int. Cl. A61M 29/00

(52) U.S. Cl. 606/200

(57) ABSTRACT

Devices, systems and methods for retrieving an intravascular filter from a body lumen are disclosed. A snare retrieval catheter in accordance with an exemplary embodiment of the present invention may include an elongated tubular member having a lumen configured to receive an elongated member. The elongated member extends axially through the lumen of the elongated tubular member, out the distal end of the catheter, and is looped-back such that both ends of the elongated member are located proximal the proximal end of the catheter. The portion of the wire extending distally from the distal end of the catheter forms an adjustable loop that can be used to ensnare a radial notch formed on the intravascular filter.

### FIELD OF THE INVENTION

The present invention relates generally to the field of intravascular filters. More specifically, the present invention pertains to retrieval mechanisms for embolic protection filters.

### BACKGROUND OF THE INVENTION

Distal protection devices such as embolic protection filters are generally placed within a body lumen, such as an artery or vein, downstream of a site where a therapeutic device will be used. Examples of procedures employing such filters include angioplasty, atherectomy, thrombectomy, and stent placement. These procedures typically involve percutaneously inserting and delivering within the artery or vein, a guidewire and embolic protection filter to a location distal a coronary lesion. Once in place, a therapeutic device such as an angioplasty or atherectomy catheter can be advanced to the site of the lesion to treat the vessel. During the procedure, embolic debris such as plaque or thrombus become dislodged from the vessel wall, and flow downstream where they are collected by the filter. After the procedure is performed, the therapeutic device, filter and captured embolic debris can be removed from the body.

### SUMMARY OF THE INVENTION

The present invention relates to systems for retrieving embolic protection filters within a vessel. In an exemplary embodiment of the present invention, a filter retrieval system may include an embolic protection filter slidably disposed along a guidewire, and a snare retrieval catheter adapted to ensnare the embolic protection filter. The embolic protection filter may comprise a filter frame, a plurality of expandable struts, and a filter mesh or membrane operatively coupled to the expandable struts. A radial notch formed on the proximal portion of the filter frame forms a collar that can be utilized by the snare retrieval catheter to retrieve the embolic protection filter from the vessel.

The snare retrieval catheter may comprise an elongated tubular member having a first lumen configured to slidably receive the guidewire, and a second lumen configured to receive an elongated member that can be actuated within the catheter to retrieve the embolic protection filter. The elongated member may include a flexible wire that extends through the second lumen of the elongated tubular member, forming a loop portion distal the distal end of the snare retrieval catheter. The size of the loop can be adjusted to ensnare the radial notch formed on the proximal portion of the embolic protection filter.

### BRIEF DESCRIPTION OF THE DRAWINGS

FIG. 1 is a perspective view of a filter retrieval system in accordance with an exemplary embodiment of the present invention;

FIG. 2 is a cross-sectional view of the snare retrieval catheter illustrated in FIG. 1, wherein the catheter is shown along line 2-2;

FIG. 3 is a plan view illustrating a guidewire inserted in a vessel distal a lesion;

FIG. 4 is a plan view of a distal stop advanced along the guidewire illustrated in FIG. 3;

FIG. 5 is a plan view of an embolic protection filter advanced along the guidewire and abutting the distal stop illustrated in FIG. 4;

FIG. 6 is a plan view of a sheath advanced about the embolic protection filter; and

FIG. 7 is a plan view of a snare retrieval catheter advanced along the guidewire and ensnaring the collapsed embolic protection filter.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

FIG. 1 is a perspective view of an exemplary embodiment of a filter system for retrieving an embolic protection filter within a blood vessel. Filter system 10 comprises a snare retrieval catheter 12, and an embolic protection filter 14 slidably disposed along a guidewire 16. As is discussed in greater detail below, the snare retrieval catheter 12 is configured to ensnare the embolic protection filter 14 and retrieve the device from blood vessel.

Embolic protection filter 14 may comprise a filter frame 18, a plurality of expandable struts 20, and a filter mesh or membrane 22 operatively coupled to the expandable struts 20. The expandable struts 20 are biased to expand in an outward direction when unconstrained radially, allowing the filter mesh or membrane 22 to expand when placed within the vessel. Several openings or pores 24 disposed on the filter mesh or membrane 22 are adapted to capture embolic debris contained in the blood while permitting the perfusion of blood through the vessel.

Filter frame 18 defines a tubular member having a proximal portion 26, a distal portion 28, and an inner lumen (not shown) configured to slidably receive the guidewire 16. The proximal portion 26 of filter frame 18 includes a radial notch 34 forming a collar that can be engaged by the snare retrieval catheter 12 to retrieve the embolic protection filter 14 from the vessel.

In the particular view depicted in FIG. 1, the embolic protection filter 14 and snare retrieval catheter 12 are shown advanced towards the distal end 30 of the guidewire 16. The snare retrieval catheter 12 is positioned proximate and proximal the embolic protection filter 14 just prior to retrieval of the device. A distal stop 32 placed at or near the distal end 30 of the guidewire 16 prevents movement of the embolic protection filter 14 beyond the distal end 30 thereof. The distal stop 32 may be formed from a tubular segment having an inner lumen that forms a friction fit when inserted over the guidewire 16. The friction fit prevents movement of the distal stop 32 along the guidewire 16 in the absence of a force exerted by the operator via a push tube or other pushing means.

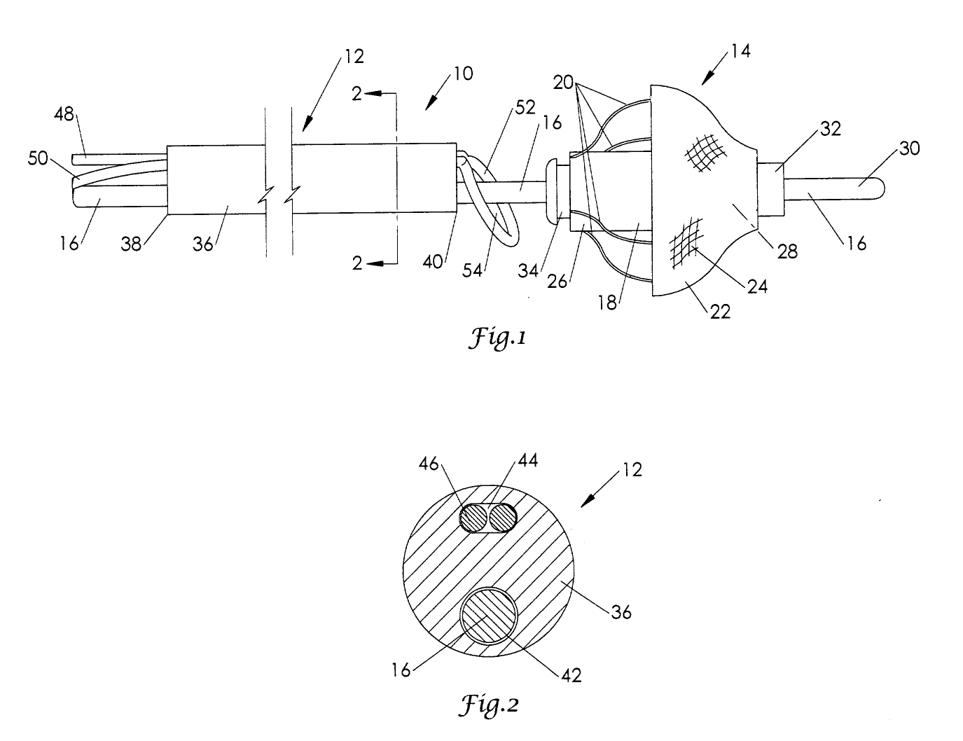

Snare retrieval catheter 12 comprises an elongated tubular member 36 having a proximal end 38 and a distal end 40. As shown in FIG. 2, the elongated tubular member 36 defines a first lumen 42 configured to slidably receive the guidewire 16, and a second lumen 44 configured to receive an elongated member 46 that can be actuated within the snare retrieval catheter 12 to retrieve the embolic protection filter 14. The first and second lumens 42, 44 extend axially through the entire length of the elongated tubular member 36, terminating at the proximal and distal ends 38, 40 thereof.

The elongated tubular member 36 may comprise a hypo-tube formed of a metal, metal-alloy or metal-polymer composite. Examples of suitable materials include stainless steel, nickel-titanium alloy, polyethylene terapthalate (PET), polytetraflouroethylene (PTFE), polyurethane, fluorinated ethylene propylene (FEP), polypropylene (PP), polyvinylchloride (PVC), polyether-ether ketone (PEEK), polyimide, polyester, polyamide, elastomeric polyamides, block polyamide/ethers, polyether block amide (PEBA), and polyetherimide (PEI).

The elongated member 46 may comprise a flexible wire or cord that extends axially through the second lumen 44, out the distal end 40 of the snare retrieval catheter 12, and is then looped-back through the second lumen 44. In the exemplary embodiment illustrated in FIG. 1, both ends 48, 50 of the elongated member 46 extend proximal the proximal end 38 of the catheter 12 at a location outside of the patient's body.

The portion of the elongated member 46 extending distally from the distal end 40 of the catheter 12 forms a loop 52, the size of which can be adjusted by moving the elongated member 46 within the second lumen 44. As shown in FIG. 1, the loop 52 is bent at an angle relative to the longitudinal axis formed by the catheter 12 such that the opening 54 formed by the loop 52 encircles the guidewire 16. The opening 54 formed by loop 52 may also be centrally disposed about a central axis defined by the guidewire 16 such that the loop 52 is concentric with the radial notch 34 formed on the filter frame 18.

To actuate the snare retrieval catheter 12, the loop 52 may be adjusted by pulling one or both of the ends 48, 50, causing the elongated member 46 to move axially within the second lumen 44. The size of the loop 52 can be adjusted to ensnare the radial notch 34 formed on the proximal portion 26 of the filter frame 18. A locking hub or other locking means (not shown) can be utilized to lock one or both of the ends 48, 50 to prevent undesired expansion or contraction of the loop 52 once the embolic protection filter 14 has been ensnared.

In some embodiments, the elongated member 46 can be formed of a metal such as stainless steel or nickel-titanium alloy, or a fibrous polymer such as polyethylene, nylon, polyester, or polyurethane. The elongated member 46 may be formed from several threads or strands woven together to form an axially stiff but radially flexible member. The structure and dimensions of the elongated member 46 can be selected to impart a particular performance characteristic, depending on the particular intravascular device to be retrieved and the particular location of the body to be traversed. For example, to retrieve an embolic protection filter from the distal vasculature, the length of the snare retrieval catheter may approach or even exceed 300 cm in length. Due to the relatively long length of the elongated member 46, buckling or kinking may result when the member 46 is compressed. Moreover, the elongated member 46 may tend to yield or even break if the tensile strength of the member 46 is insufficient to transfer the load of the embolic protection filter 14. Accordingly, the dimensions and/or materials of the elongated member 46 can be selected to account for the tensile, compressive, and torsional stresses resulting from the advancement and/or retraction of the elongated member 46 within the second lumen 44.

In certain embodiments, the elongated member 46 may be formed of a radiopaque material (e.g. gold, platinum, tungsten, tantalum), or may include a radiopaque additive (e.g. barium sulfate, barium subcarbonate), allowing the operator to determine the location of the elongated member 46 within the body. In use, a contrast media can be injected into the vessel, and a fluoroscopic monitor located outside of the patient's body can be utilized to determine the precise location of the elongated member 46 within the vessel.

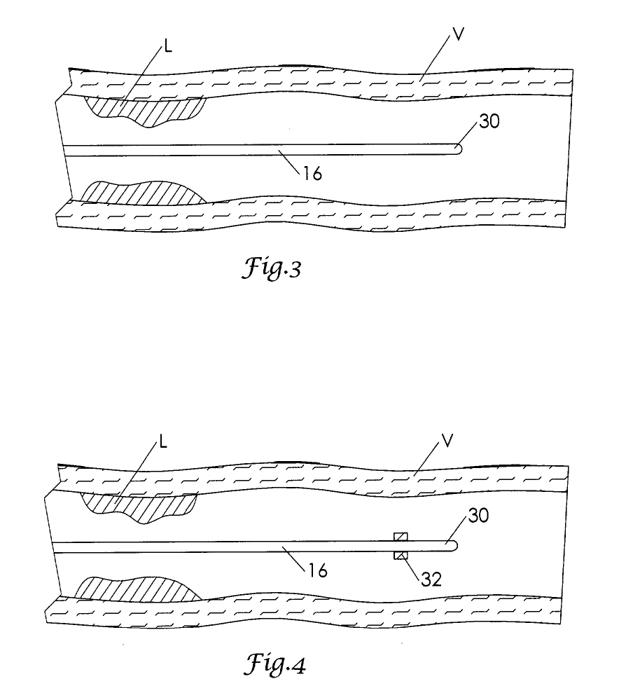

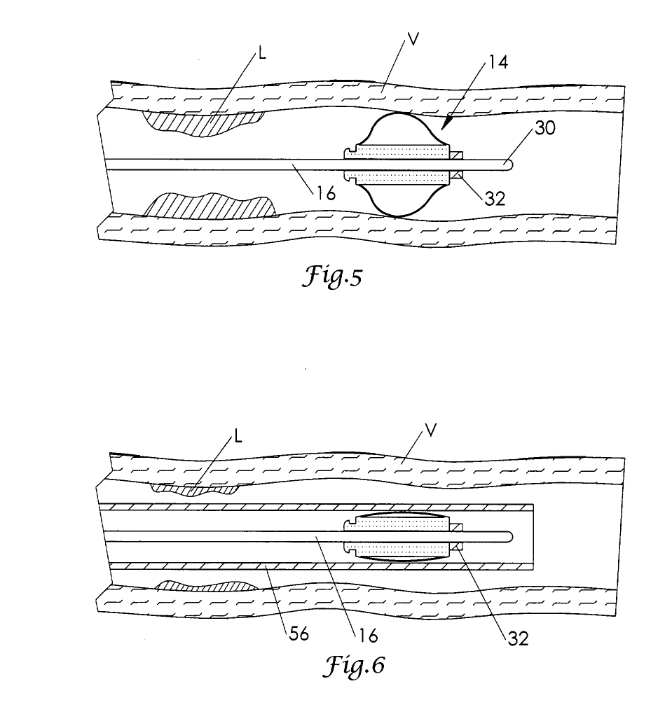

FIGS. 3-7 depict a retrieval method using the snare retrieval catheter 12 and embolic protection filter 14 described above in the context of a therapeutic procedure such as percutaneous transluminal coronary angioplasty (PTCA). In a first position illustrated in FIG. 3, a guiding device such as a guidewire 16 can be advanced through a vessel V, and placed at a desired location distal a lesion L. Once positioned, a distal stop 32 can be placed at or near the distal end 30 of the guidewire 16, as shown in FIG. 4. The embolic protection filter 14 can then be advanced along the guidewire 16 and placed at a location proximal the distal stop 32, as shown in FIG. 5. The distal stop 32 prevents the operator from advancing the embolic protection filter 14 beyond the distal end 30 of the guidewire 16. An angioplasty procedure can then be conducted upstream of the embolic protection filter 14 to treat the lesion L. At the conclusion of the therapeutic procedure, a sheath 56 can be advanced along the guidewire 16 to collapse the embolic protection filter 14 and its contents therein, as shown in FIG. 6.

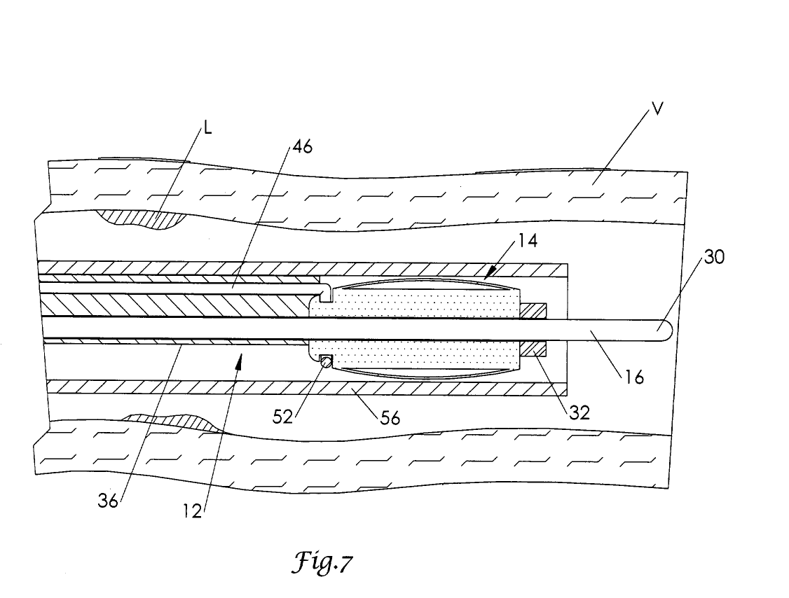

Once the embolic protection filter 14 has been collapsed within the sheath 56, the snare retrieval catheter 12 can be advanced along the guidewire 16 and positioned such that the loop 52 is disposed at least in part about the radial notch 34. Holding the snare retrieval catheter 10 stationary, the operator can then retract the elongated member 46 proximally through the second lumen 44 to reduce the size of the loop 52 and ensnare the embolic protection filter 14, as shown in FIG. 7. The sheath 56, snare retrieval catheter 12, and captured embolic protection filter 14 can then be removed from the body.

It should be understood that other intravascular devices can be retrieved using the devices described herein. For example, although a cartridge-type embolic protection filter is illustrated, a basket, umbrella, or hoop-type embolic protection device can be retrieved using the devices and methods described above. Moreover, intravascular devices for use in other areas of the body (e.g. the vena cava) can be retrieved using the devices and methods described herein.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

What is claimed is:

1. A system for retrieving an intravascular filter within a blood vessel, comprising:
a guidewire;
an embolic protection filter slidably disposed along the guidewire; and
a snare retrieval catheter adapted to ensnare the embolic protection filter.

2. The system of claim 1, wherein the snare retrieval catheter further comprises:
an elongated tubular member having a proximal end, a distal end, a first lumen configured to slidably receive the guidewire, and a second lumen configured to receive an elongated member, said elongated member having a first end, a second end, and a loop portion extending distally of the distal end of said elongated tubular member, said loop portion forming an adjustable snare that can be sized within the blood vessel to retrieve the embolic protection filter.

3. The system of claim 1, wherein said embolic protection filter comprises a filter frame, a plurality of expandable struts, and a filter mesh or membrane operatively coupled to said plurality of expandable struts.

4. The medical retrieval device of claim 3, wherein said filter frame includes a radial notch.

5. The medical retrieval device of claim 2, wherein said elongated member is a wire.

6. The medical retrieval device of claim 2, wherein said elongated member is a cord.

7. The medical retrieval device of claim 2, wherein the loop portion is bent at an angle relative to the longitudinal axis formed by the elongated tubular member.

8. The medical retrieval device of claim 2, wherein the loop portion encircles the guidewire.

9. The medical retrieval device of claim 2, wherein the adjustable snare is centrally disposed about a central axis defined by the guidewire.

10. The medical retrieval device of claim 1, further comprising a distal stop disposed about the guidewire.

11. A system for retrieving an intravascular filter within a blood vessel, comprising:
a guidewire;
an embolic protection filter comprising a filter frame slidably disposed along the guidewire, said filter frame including a radial notch; and
a snare retrieval catheter adapted to ensnare the radial notch on the embolic protection filter, said snare retrieval catheter comprising:
an elongated tubular member having a proximal end, a distal end, a first lumen configured to slidably receive the guidewire, and a second lumen configured to receive an elongated member, said elongated member having a first end, a second end, and a loop portion extending distally of the distal end of said elongated tubular member, said loop portion forming an adjustable snare that can be sized within the blood vessel to retrieve the embolic protection filter.

12. A method of retrieving an embolic protection filter disposed along a guidewire in a body lumen, comprising the steps of:
providing a snare retrieval catheter along the guidewire proximal the embolic protection filter, said snare retrieval catheter including an elongated member having a loop portion forming an adjustable snare;
advancing the loop portion of said snare retrieval catheter about the embolic protection filter; and
adjusting the size of the loop portion to ensnare the embolic protection filter.

13. A method of retrieving an embolic protection filter disposed along a guidewire in a body lumen, comprising the steps of:
providing an embolic protection filter having a filter frame, a plurality of expandable struts, and a filter mesh or membrane operatively coupled to the expandable struts, said filter frame including a radial notch;
providing a snare retrieval catheter along the guidewire proximal the embolic protection filter, said snare retrieval catheter including an elongated member having a loop portion forming an adjustable snare;
advancing the loop portion of said snare retrieval catheter about the radial notch formed on the embolic protection filter;
engaging the loop portion about the radial notch to ensnare the embolic protection filter; and
removing the snare retrieval catheter and ensnared embolic protection filter from the body lumen.

14. A medical retrieval device for retrieving an intravascular filter within a blood vessel, said device comprising:
an elongated tubular member having a proximal end, a distal end, a first lumen, and a second lumen; and
an elongated member disposed within the second lumen having a first end, a second end, and a loop portion extending distally of the distal end of said elongated tubular member, said loop portion forming an adjustable snare that can be sized within the blood vessel to retrieve the intravascular filter.

15. The medical retrieval device of claim 14, wherein said intravascular filter is an embolic protection filter.

16. The medical retrieval device of claim 15, wherein said embolic protection filter comprises a filter frame, a plurality of expandable struts, and a filter mesh or membrane operatively coupled to said plurality of expandable struts.

17. The medical retrieval device of claim 16, wherein said filter frame includes a radial notch.

18. The medical retrieval device of claim 14, wherein said elongated tubular member is formed of hypodermic tubing.

19. The medical retrieval device of claim 14, wherein said elongated member is a wire.

20. The medical retrieval device of claim 14, wherein said elongated member is a cord.

21. The medical retrieval device of claim 14, wherein said elongated member is formed of a metal.

22. The medical retrieval device of claim 21, wherein said metal is stainless steel.

23. The medical retrieval device of claim 14, wherein said elongated member is formed of a fibrous polymer.

24. The medical retrieval device of claim 14, wherein said elongated member is radiopaque.

25. The medical retrieval device of claim 14, wherein the first lumen of said elongated tubular member is configured to slidably receive a guidewire.

26. The medical retrieval device of claim 25, wherein the loop portion of said elongated member is bent at an angle relative to the longitudinal axis formed by the elongated tubular member.

27. The medical retrieval device of claim 25, wherein the loop portion encircles the guidewire.

28. The medical retrieval device of claim 25, wherein the loop portion is centrally disposed about a central axis defined by the guidewire.

29. The medical retrieval device of claim 25, further comprising a distal stop disposed about the guidewire.

30. A medical retrieval device for retrieving an intravascular filter within a blood vessel, said device comprising:
an elongated tubular member having a proximal end, a distal end, a first lumen configured to slidably receive a guidewire, and a second lumen configured to receive an elongated member;
said elongated member having a first end, a second end, and a loop portion extending distally of the distal end of said elongated tubular member, said loop portion forming an adjustable snare that can be sized within the blood vessel to retrieve the intravascular filter.

31. The medical retrieval device of claim 30, wherein said intravascular filter is an embolic protection filter.

32. The medical retrieval device of claim 31, wherein said embolic protection filter comprises a filter frame, a plurality of expandable struts, and a filter mesh or membrane operatively coupled to said plurality of expandable struts.

33. The medical retrieval device of claim 32, wherein said filter frame includes a radial notch.

34. The medical retrieval device of claim 30, wherein said elongated tubular member is formed of hypodermic tubing.

35. The medical retrieval device of claim 30, wherein said elongated member is a wire.

36. The medical retrieval device of claim 30, wherein said elongated member is a cord.

37. The medical retrieval device of claim 30, wherein said elongated member is formed of a metal.

38. The medical retrieval device of claim 37, wherein said metal is stainless steel.

39. The medical retrieval device of claim 30, wherein said elongated member is formed of a fibrous polymer.

40. The medical retrieval device of claim 30, wherein said elongated member is radiopaque.

41. The medical retrieval device of claim 30, wherein the loop portion is bent at an angle relative to the longitudinal axis formed by the elongated tubular member.

42. The medical retrieval device of claim 30, wherein the loop portion encircles the guidewire.

43. The medical retrieval device of claim 30, wherein the loop portion is centrally disposed about a central axis defined by the guidewire.

44. The medical retrieval device of claim 30, further comprising a distal stop disposed about the guidewire.
